(19) United States
(12) Patent Application Publication
(10) Pub. No.: US 2026/0014358 A1
(43) Pub. Date: Jan. 15, 2026

(54) THERMALLY REGULATED TRANSDERMAL DRUG DELIVERY SYSTEM

(71) Applicant: Morningside Venture Investments Limited, Monaco (MC)

(72) Inventors: Patrick H. RUANE, Dublin, CA (US); Jackie Joe HANCOCK, Berkeley, CA (US); Anubhav ARORA, Hayward, CA (US)

(21) Appl. No.: 19/275,863

(22) Filed: Jul. 21, 2025

Related U.S. Application Data
(63) Continuation of application No. 17/293,570, filed on May 13, 2021, now Pat. No. 12,397,141.
(60) Provisional application No. 62/768,768, filed on Nov. 16, 2018.

(51) Int. Cl. A61M 37/00; A61K 31/465

(52) U.S. Cl. CPC A61M 37/00; A61K 31/465; A61M 2037/0007; A61M 2205/055; A61M 2205/3606; A61M 2205/368; A61M 2205/8206

(57) ABSTRACT

Described herein are drug and other active agent transdermal delivery systems and devices to deliver active agents to skin or mucosa of a subject, and methods of delivering such active agents. In particular, systems, methods, and devices are described that control the concentration and timing of active agent delivery. Such systems or devices may include a transdermal membrane and a temperature control element for heating and/or cooling a portion of the transdermal delivery system to provide pulsatile active agent delivery through the transdermal membrane.

### CROSS REFERENCE TO RELATED APPLICATIONS

This application is a continuation of U.S. patent application Ser. No. 17/293,570, filed May 13, 2021, titled “THERMALLY REGULATED TRANSDERMAL DRUG DELIVERY SYSTEM,” now U.S. Patent Application Publication No. US20220001158-A1, which is the U.S. National Stage under 35 U.S.C. 371 of International Application No. PCT/US2019/061761, filed Nov. 15, 2019, titled “THERMALLY REGULATED TRANSDERMAL DRUG DELIVERY SYSTEM,” now International Application Publication No. WO 2020/102695, which claims priority to U.S. Provisional Application No. 62/768,768, filed Nov. 16, 2018, and titled “Thermally Regulated Transdermal Drug Delivery System,” the entirety of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure is related generally to delivery of drugs and other active agents to a subject. More specifically, this disclosure relates to delivery of drugs and other active agents to skin or mucosa of a subject.

### BACKGROUND

Cigarette smoking causes more than 480,000 deaths each year in the United States alone. Smoking increases the risk of coronary heart disease, stroke, lung cancer and other diseases up to 25 times. Despite the availability of pharmaceutical agents and various patches to help people stop smoking, many people are unable to give up smoking. This is mostly due to the strong cravings for smoking that smokers feel.

Further, approximately one million people in the United States alone have Parkinson's disease. Parkinson's disease is a degenerative disease of the nervous system with no known cure. The symptoms of Parkinson's disease include tremor, joint rigidity, balance problems, and slow movement. The symptoms get progressively worse over time.

Various systems have been developed to deliver pharmaceutical agents to a subject, such as those who wish to stop smoking or who suffer from Parkinson's disease. However, these existing systems suffer from various limitations and problems. For example, injections of a pharmaceutical drug is painful. Orally ingested drugs may not be available when needed and/or may dissolve in the acid of the stomach.

Transdermal drug delivery, i.e., using the skin of the body to deliver pharmaceutical agents, can be advantageous, as transdermal delivery prevents pharmaceutical agents from being digested in the stomach and eliminates pain from direct injection. However, the barrier function of the skin can make it difficult to get pharmaceutical drugs transported across the skin and into the body. Thus, for example, in passive transdermal drug delivery, a drug delivery patch is placed on the skin, and the rate at which the patient receives the drug is limited by how quickly (or slowly) the drug can diffuse across the skin. Additionally, such passive transdermal delivery systems often require frequent replacement of the transdermal patch. Further, passive transdermal systems often do not provide customizable drug delivery, making them less suitable for clinical applications that require a fast or controlled onset, such a quick delivery of nicotine to decrease cigarette cravings or a timely delivery of Parkinson's drugs to counteract Parkinson's symptoms.

Described herein are systems and methods to address limitations of existing transdermal drug delivery systems.

### SUMMARY OF THE DISCLOSURE

Described herein are drug and other active agent delivery systems and devices for delivering active agents to skin or mucosa of a subject, and methods of delivering such active agents, and in particular, for controlling the timing and amount of drug and other active agent delivery.

In general, in one embodiment, a transdermal delivery system includes an active agent integrated with or fluidically connected to a transdermal membrane and a temperature control element. The transdermal membrane is configured to allow the active agent to flow therethrough to skin of a subject. The temperature control element is configured to heat and/or cool a portion of the transdermal delivery system so as to provide pulsatile delivery of the active agent through the transdermal membrane.

This and other embodiments can include one or more of the following features. The temperature control element can include an electromagnetic energy source. The temperature control element can include a resistive element. The temperature control element can include inductive coil or an electromagnet. The temperature control element can include a coolant or heat sink. The portion of the transdermal membrane can include a polymer configured to be heated or cooled to thereby change active agent flow. The portion of the transdermal membrane can include a glass transition polymer configured to be heated or cooled to thereby change active agent flow. The portion of the transdermal membrane can include a magnetic nanoparticle configured to be heated or cooled to thereby change active agent flow. The active agent can include nicotine or a nicotine agonist. The active agent can include a Parkinson's disease treatment. The active agent can include at least one of benztropine, carbidopa, dopamine, a dopamine analog, a dopamine antagonist, a dopamine agonist, entacapone, levodopa (L-dopa), pramipexole, rasagiline, ropinirole, rotigotine, safinamide, selegiline, both carbidopa and levodopa, trihexyphenidyl and tolcapone. The transdermal delivery system can further include an adhesive in the transdermal membrane configured to adhere the transdermal membrane to the subject. The transdermal delivery system can further include an adhesive in the transdermal membrane, and the adhesive can contain the active agent. The transdermal delivery system can further include a temperature sensor configured to measure the temperature of at least one of the temperature control element, the portion of the transdermal delivery system, or the transdermal membrane. The transdermal delivery system can further include a reservoir of active agent. The transdermal delivery system can further include a power source configured to provide power to the temperature control element. The transdermal delivery system can further include a circuit board. The transdermal delivery system can further include a microcontroller configured to control delivery of a stimulus from the temperature control element. The transdermal delivery system can further include a communication element configured to receive or transmit data and the communication element can include Bluetooth or WiFi.

In general, in one embodiment, a method of transdermally delivering an active agent, includes (1) actively heating or cooling a portion of a transdermal delivery system for a first amount of time; and (2) after the first amount of time, actively heating or cooling the portion of the transdermal delivery system for a second amount of time. The actively heating or cooling steps provide pulsatile delivery of active agent from a transdermal membrane of the transdermal delivery system to skin of a patient.

This and other embodiments can include one or more of the following features. The heating or cooling steps can include delivering heat to the portion of the transdermal delivery system. The actively heating or cooling steps can include removing heat from the portion of the transdermal delivery system. At least one of the actively heating or cooling steps can include delivering electromagnetic radiation to the portion of the transdermal delivery system. The method can further include repeating the actively heating or cooling steps at least once. The method can further include repeating the actively heating or cooling steps at least twice. The portion of the transdermal delivery system can be increased in temperature by at least 4° C. during the actively heating and cooling steps. The method can further include actively heating skin of the subject adjacent the transdermal membrane by at least 3° C. during the actively heating or cooling steps. At least one of the actively heating or cooling steps can be performed while the subject is sleeping.

In general, in one embodiment, a transdermal delivery system includes an active agent, a transdermal membrane, and a temperature control element. The active agent is integrated with or fluidically connected to a transdermal membrane. The transdermal membrane is configured to allow the active agent to flow therethrough to skin of a subject. The temperature control element is configured to provide a stimulus to a portion of the transdermal delivery system so as to provide pulsatile delivery of the active agent through the transdermal membrane to the skin of the subject.

This and other embodiments can include one or more of the following features. The system can further include a reactive material configured to prevent the active agent from flowing across the transdermal membrane until the stimulus is applied. The reactive material can include an epoxy, a polyethylene, a polymethacrylate, a polypropylene, a polypropylene glycol, a polyvinylacetate, a polystyrene, a polytetrafluoroethylene, a poly (bisphenol A carbonate), a poly (ethylene terephthalate), a polylactic acid (PLA), a polyglycolic acid (PGA), or a polyurethane. The reactive material can include a polymer, a hydrogel, a solvent, gold covered nanoparticles, or magnetic nanoparticles.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a transdermal delivery system with temperature control for controlling active agent delivery to a subject.

FIG. 2 shows another transdermal active agent delivery system with temperature control for controlling active agent delivery to a subject.

FIG. 3 shows a transdermal delivery system with temperature control and a complementary transdermal membrane for controlling active agent delivery to a subject.

FIG. 4 shows results of nicotine delivery after heating a matrix-type transdermal membrane to deliver nicotine through the transdermal membrane.

### DETAILED DESCRIPTION

Transdermal delivery systems, devices, and methods are described herein for delivering an active agent, such as a drug, to a subject. For example, described herein are transdermal delivery systems configured to control delivery of one or more active agents to a skin or mucosal surface of a subject and methods of using such systems. The active agent may be any agent useful for furnishing pharmacological activity or other effect in the diagnosis, cure, mitigation, treatment, or prevention of a disorder, a disease, a symptom, a syndrome and/or to affect any structure or any function of a human or other body. The active agent may act on the skin or mucosal surface and/or move into the blood stream, where it may travel systemically.

In some embodiments, the transdermal delivery systems described herein can be used to provide an active agent at a steady state level in the bloodstream. For example, the transdermal delivery systems can be used to provide an extended release medicine to provide a steady state of relief to manage chronic pain. In other embodiments, the transdermal drug delivery systems described herein can be used to provide an active agent such that the level of active agent varies in the bloodstream change over time. For example, the transdermal delivery systems can be used to deliver nicotine at particular times for smoking cessation. A habitual smoker who is trying to give up smoking can have very powerful cravings for cigarettes at particular times, such as upon waking up after a night's sleep and after eating a meal. Since a dose of nicotine can help manage intense cravings, it may be advantageous to give such a person a dose of nicotine only at certain times, e.g., when the cravings are strongest.

In general, the transdermal delivery systems described herein include an active agent integrated with or fluidically connected to a transdermal membrane, a transdermal membrane configured to allow active agent to flow therethrough, and a temperature control element configured to stimulate a portion of the transdermal delivery system (e.g., a transdermal membrane or an active agent therein) so as to control the timing and/or amount of delivery of the active agent through the transdermal membrane. In some embodiments, the temperature control element is a heating and cooling element configured to heat and/or cool a portion of the transdermal delivery membrane so as to provide pulsatile delivery of the active agent through the transdermal membrane. The transdermal systems described herein can be configured to control an amount and a timing of active agent delivery through the transdermal delivery system.

The transdermal systems described herein can be configured to release an active agent through the transdermal membrane to a subject in a pulsatile flow. As a result, the flux (the amount of active agent that crosses the skin per unit area per unit time) can be controlled and tuned up and down. For example, the flux can be controlled so as to deliver active agent for 1 delivery cycle (with 1 peak and 1 valley), 2 delivery cycles, 3 delivery cycles, or more than 3 delivery cycles. Each delivery cycle may be from a few minutes to many hours or even a day or more. Further, different delivery cycles may advantageously last for different lengths of time. When fluid is delivered during a delivery cycle, it may be referred to a pulse of active agent delivery. The transdermal delivery systems described herein can control the period of time over which a desired amount of an active agent flows across a transdermal membrane for any given cycle.

In any of the delivery systems described herein, the concentration of active agent in the transdermal membrane available for delivery to a subject may be alternately increased and decreased, allowing for alternately increased and decreased flow (including down to no flow) of active agent across the transdermal membrane to the subject. In some embodiments, the transdermal systems described herein may be able to alternately heat and cool a portion of the transdermal delivery system to increase and decrease the concentration of active agent in the transdermal membrane.

The drug delivery systems and methods described herein can heat, cool, or otherwise stimulate the skin to control the flux of active agent therethrough. That is, once flow of active agent has reached skin of a subject, flow of the active agent across the skin of the subject is modulated by characteristics of the skin, such as skin composition and skin temperature. In some embodiments, a portion of the subject's skin, such as skin adjacent to the transdermal membrane, may be stimulated, such as by heating and cooling, to modulate active agent delivery across the skin.

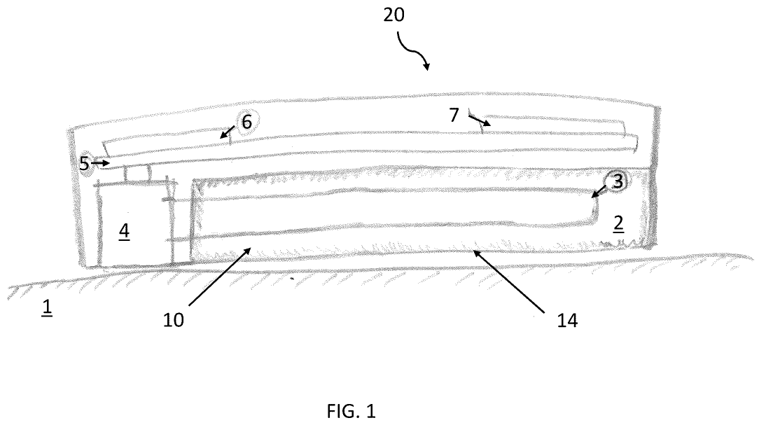

FIG. 1 shows a transdermal delivery system 20 with temperature control element 3, power source 4, and transdermal membrane 10. Active agent 2 is integrated with transdermal membrane 10. Adhesive matrix 14 adheres transdermal delivery system 20 to skin 1 of the subject. Transdermal membrane 10 is configured to allow the active agent 2 in the transdermal membrane 10 to flow therethrough to skin 1 of the subject. Power source 4 is configured to provide power to the temperature control element 3 and may be a rechargeable or non-rechargeable battery such as a lithium metal, lithium ion, nickel metal hydride, or nickel cadmium battery. If rechargeable, power source 4 may be rechargeable using a wired or wireless mechanism, such as using wireless inductive recharging from a smart phone.

Temperature control element 3 can be positioned at least partially within transdermal membrane 10. The temperature control element 3 may include one or more resistive heating elements, infrared light sources, or chemicals for exothermic chemical reactions. In some embodiments, temperature control element 3 is configured to generate heat (e.g., kinetic energy) or other energy. Further, temperature control element 3 can deliver heat, coolant, or other energy to the transdermal membrane 10 and thereby control (increase, decrease or maintain) a temperature of the transdermal membrane 10 and/or active agent 2. The temperature control element 3 may increase temperature of a portion of the transdermal membrane 10 (and/or the active agent 2) by at least 1° C., at least 2° C., at least 3° C., at least 4° C., at least 5° C., at least 6° C., at least 7° C., at least 8° C., at least 9° C. or at least 10° C. or by not more than 1° C., not more than 2° C., not more than 3° C., not more than 4° C., not more than 5° C., not more than 6° C., not more than 7° C., not more than 8° C., not more than 9° C., or not more than 10° C., or any value between these numbers (such as at by at least 2° C. and not more than 5° C.) by the applied heat or other energy. Further, the applied heat or other energy from the temperature control element 3 can act on the transdermal membrane 10 (and/or a component in the transdermal membrane 10, such as the active agent 2) to allow or increase active agent delivery through transdermal membrane 10 to skin 1. For example, the applied heat or other energy can increase active agent diffusion and movement within transdermal membrane 10 and/or can increase the pore size of the transdermal membrane 10 to enhance flow therethrough.

In some embodiments, energy delivery to a portion of the transdermal delivery system 20, such as to the transdermal membrane 10, may alternate between two (or more) conditions, such as an “on” condition and an “off” condition. For example, a first delivery condition may have higher level of heat delivery (e.g., a delivery at a higher temperature and/or delivery for a longer time), and a second delivery condition may have a lower level (including no) heat delivery (e.g., lower temperature and/or for a longer time). A higher level of heat delivery can be sufficiently high so as to enable flow of a sufficient amount of active agent across the transdermal membrane 10 to skin 1 to have a therapeutic, prophylactic, or other effect. In contrast, a lower heat delivery state can be sufficiently low (or be off) such that there is insufficient (or no) flow of active agent across transdermal membrane 10 and therefore no therapeutic or prophylactic effect. In some embodiments, the transdermal delivery system 20 may alternate repeatedly between higher and lower heat delivery conditions to create pulsatile active agent delivery (e.g., a plurality of pulses of active agent delivery). For example, the transdermal delivery system 20 may produce 1 pulse, 2 pulses, 3 pulses, 4 pulses, 5 pulses, 6 pulses, 7 pulses, 8 pulses, 9 pulses, or 10 pulses or more than 10 pulses.

In some embodiments, a lower (or no) heat delivery state may result from temperature control element 3 being off or inactive, thereby reducing or eliminating heat delivery to a portion of the transdermal delivery system 20. In some embodiments, temperature control element 3 may be configured (e.g., may have a refrigerant or act as a heat sink) to actively decrease a temperature of a portion of the transdermal delivery system 20. The lower (or no heat) delivery or active cooling by the temperature control element 3 may reduce the temperature of a portion of transdermal delivery system 20 (elative to the temperature of that portion of the transdermal delivery system 20 or relative to the temperature of that portion of the transdermal delivery system 20 that has previously been heated) by at least 1° C., at least 2° C., at least 3° C., at least 4° C., at least 5° C., at least 6° C., at least 7° C., at least 8° C., at least 9° C., or at least 10° C. or by not more than 1° C., not more than 2° C., not more than 3° C., not more than 4° C., not more than 5° C., not more than 6° C., not more than 7° C., not more than 8° C., not more than 9° C., or not more than 10° C. or any value between these numbers (such as by at least 2° C. and not more than 5° C.). In some embodiments, the temperature of a portion of the transdermal delivery system 20 in place on the user's skin may be about 30° C. (e.g., between 29.5° C. and 30.5° C.), about 31° C., about 32° C., about 33° C., about 34° C., about 35° C., about 36° C., about 37° C., about 38° C., or about 39° C. (e.g., +/−0.5° C.). As the temperature of the user's skin may be cooler than the user's body or blood temperature (e.g., about 32° C., about 33° C., or about 34° C. rather than about 36° C. or about 37° C.), the baseline temperature of part of the transdermal delivery system 20 in place on the user may be about 32° C., about 33° C., or about 34° C., and the transdermal delivery system 20 may be configured to not deliver active agent at such temperatures.

In some embodiments, the transdermal delivery system 20 may be configured to heat or cool the user's skin instead of, or in addition to, heating or cooling a portion of the transdermal delivery system 20. Such heating or cooling may alter active agent delivery through the skin, such as allowing or reducing active agent delivery. Thus, the temperature control element 3 may be configured to deliver heating or cooling or other energy to skin 1 of the subject to heat or cool the skin 1. The temperature control element 3 may deliver heating or cooling or other energy to skin 1 indirectly (e.g., through conduction, such as a heated or cooled transdermal membrane) directly or (e.g., via infrared energy delivery to heat the skin). Heating of the skin may allow the flux (amount of active agent that crosses the skin per unit area per unit time) to exceed that which is typically observed in transdermal delivery (e.g., in passive transdermal delivery systems) and/or that which is achieved without heating. This increased flux may be useful, for example, for getting active agents that are not readily deliverable by other transdermal systems delivered across the skin or for controlling pulses of active agent delivery. In some embodiments, both the transdermal membrane 10 and the skin 1 can be heated in response to the applied heat or other energy.

In some embodiments, the upper limit, lower limit, or range of desired temperatures for the portion of transdermal delivery system 20 to be heated or cooled (and/or the temperature of the skin) may be tuned to the particular system for desired efficacy, comfort, and safety. That is, too high of a temperature may be uncomfortable or may burn a subject. Too low of a temperature may not facilitate sufficient delivery of a desired amount of active agent. For example, a system 20 configured for delivering a larger amount of an active agent through the front of the arm (dorsal arm skin) may have a higher maximum temperature than a system 20 for delivering a smaller amount of an active agent through the neck. The temperature of skin 1 may be heated so that it is at least 32° C., at least 33° C., at least 34° C., at least 35° C., at least 36° C., at least 37° C., at least 38° C., at least 39° C., or at least 40° C. or to a temperature not more than 40° C., 39° C., 38° C., 37° C., 36° C., 35° C., or 34° C. or any temperature in between these numbers. The temperature of skin 1 may be increased by at least 1° C., at least 2° C., at least 3° C., at least 4° C., at least 5° C., at least 6° C., at least 7° C., at least 8° C. or by not more than 1° C., not more than 2° C., not more than 3° C., not more than 4° C., not more than 5° C., not more than 6° C., or not more than 7° C., or any value between these numbers (such as at by least 2° C. and by not more than 5° C.) by the applied heat or other energy.

The transdermal delivery system 20 may be configured to provide pulsatile delivery of active agent. Pulsatile delivery includes an “on” period of active agent delivery and an “off” period without or with a low level of active agent delivery. Temperature control element 3 may alternate between a period of heating and a period of non-heating (or cooling). Alternating a period of heating for delivering active agent with a period of non-heating (or cooling) may lead to active agent delivery with 1 pulse, 2 pulses, 3 pulses, 4 pulses, or 5 or more than 5 pulses of active agent delivery for pulsatile delivery of the active agent 2. A delivery window during which a therapeutically effective or otherwise desired dosage of an active agent is delivered from the transdermal delivery system 20 may last at least 10 minutes, at least 30 minutes, at least 1 hour, at least 2 hours, at least 3 hours, at least 4 hours, at least 6 hours, at least 8 hours, at least 10 hours, at least 12 hours, or at least 18 hours, or less than 10 minutes, less than 30 minutes, less than 1 hour, less than 1 hour, less than 2 hours, less than 3 hours, less than 4 hours, less than 6 hours, less than 8 hours, less than 10 hours, less than 12 hours, or less than 18 hours. An “off” period during which active agent delivery is “off” (or an ineffective or untherapeutic amount of active agent is delivered) may last at least 10 minutes, at least 30 minutes, at least 1 hour, at least 2 hours, at least 3 hours, at least 4 hours, at least 5 hours, at least 6 hours, at least 10 hours, at least 12 hours, at least 18 hours, at least 1 day or less than 10 minutes, less than 30 minutes, less than 1 hour, less than 2 hours, less than 3 hours, less than 4 hours, or less than 6 hours, less than 10 hours, less than 12 hours, less than 18 hours or less than 1 day or anything in between these amounts. In some embodiments, active agent delivery is “off” most of the time when a subject is planning on sleeping and then is on before the subject plans to wake up, such as one hour before the subject plans to wake up. Active agent delivery may be “on” for several pulses based on a subject's planned activities such as before the subject plans to wake up (e.g., starting one hour before), at a planned lunch time (e.g., starting 30 minutes before) and at a planned dinner time (e.g., starting 30 minutes before). The amount of active agent delivery during “on” periods may be substantially the same for each period or may be different for the different periods. For example, greater amounts of active agent may be delivered during the wake up period than at the lunch period.

FIG. 1 also shows flexible circuit board 5 and microcontroller 6 in transdermal delivery system 20 useful for controlling the active agent delivery system. Microcontroller 6 may include a processor, memory, and input/output peripheral for controlling transdermal “on” and “off” parameters. Microcontroller 6 be preprogrammed to control temperature control element 3 for turning heat or other energy on and off. Microcontroller 6 may control time of heating/no (or little) heating for temperature control element 3 as well as the number of pulses of heat/no (or little) heat. Microcontroller 6 may also control the temperature of heating/no (or little) heating. The transdermal delivery system 20, such as temperature control element 3 or transdermal membrane 10, may include a temperature sensor, such as a thermometer for measuring temperature. The temperature sensor may provide feedback to the system to stop (or reduce) heating or to start heating once a portion of the system reaches a particular temperature. The limit may be useful for limiting an amount of active agent delivery (e.g., to prevent an overdose or more active agent than desired) or to prevent discomfort or prevent a burn to the subject.

FIG. 1 also shows communication module 7 in transdermal delivery system 20. Communication module 7 may be configured to connect and communicate with an external data source, such as a smart phone, a tablet, a computer, a server, or the internet (a TCP/IP network). Communication may be by wire or may be wireless such as using Bluetooth, Bluetooth Low Energy, WiFi, WiMAX, or Zigbee technology. Communication module 7 may send data from the transdermal delivery system 20 to the external source, such as data about transdermal membrane usage, one or more temperatures of one or more portions of transdermal delivery system 20, or duration of particular temperatures in one or more portions of the transdermal delivery system 20. Communication module 7 may receive data from a user, such as data for controlling: active agent delivery time and duration, temperature changes and duration of heating, and/or number of active agent delivery pulses. In some embodiments, the user may send signals to communication module 7 from a smart phone.

The transdermal membrane 10 may be adhered or configured to adhere to the skin 1 or mucosal surface of a subject, such as through a removable adhesive matrix 14. In some embodiments, the adhesive matrix 14 may be sufficiently strong such that it is able to hold the transdermal delivery system 20 in place on skin 1 with or without a band or other fastener. The adhesive matrix 14 may include an acrylate, a methacrylate, or an epoxy diacrylate for adhering. The adhesive matrix 14 may be configured to substantially seal the transdermal membrane 10 of the system to the subject's skin or mucosa. The adhesive matrix 14 may be configured to minimize or prevent significant entry of air or contaminants from the environment to the transdermal membrane 10. In one embodiments, the adhesive matrix 14 may be configured to prevent a solvent (e.g., in the transdermal membrane) from unwanted evaporation during transdermal system use. In some embodiments, the adhesive matrix 14 contains the active agent 2. For example, active agent 2 may be dispersed or dissolved in part or all of the adhesive matrix 14. The adhesive matrix 14 may hold part(s) of the transdermal system 20 together, and/or contain the active agent 2 and/or hold the transdermal system 20 to the subject.

FIG. 1 also shows flexible circuit board 5 and microcontroller 6 for controlling the active agent delivery system. Microcontroller 6 may be preprogrammed to control periods of heating/no heating of a portion of the transdermal delivery system 20. Microcontroller 6 may also or instead be programmed to control periods of heating/no heating from received instructions, such as from a user interface on the transdermal delivery system 20 or from an external communication, such as instructions delivered using communication module 7.

By controlling periods and levels of heating/no heating (cooling), a pulsatile delivery can be achieved from the transdermal membrane 10. The control mechanisms described herein allow the delivery of the active agent 2 to be controlled to meet the clinical need for variable active agent delivery, such as increasing therapeutic or other effect when a symptom being targeted is problematic and not at times when the symptom is not as problematic. For example, smokers often experience a craving for a cigarette at particular times, such as intense cravings when first awaking after sleep. A controlled dose of nicotine given to a subject at the right time may control such a craving and keep a person from smoking upon waking. Using the transdermal delivery system 20 with heating and cooling as described herein, for example, allows a smoker to receive a nicotine dose when their cravings are normally particularly troublesome. A dose of active agent 2 may be given when needed or in anticipation of a need. A dose of the active agent 2 may be given to a subject when a subject is awake or when they are asleep. For example, some symptoms or health issues (e.g., cigarette cravings, heart attacks, or migraine headaches) can be especially troublesome in the morning or upon awakening. Using the transdermal delivery systems described herein, a dose of the active agent 2 can be started before the subject plans to wake up, such as at least 15 minutes, at least 30 minutes, at least 45 minutes, at least 1 hour, at least 2 hours, or at least 3 hours before the subject plans to wake up.

Additionally, by tuning the properties of the transdermal system 20 so that delivery of the active agent 2 is lessened or essentially non-existent in the absence of applied heat, no or subclinical levels of active agent dosages can be delivered, effectively turning off the active agent effects in those periods. This may be achieved with an active agent/excipient/matrix adhesive such that diffusion of the active agent is minimal at unheated temperatures (e.g., 32° C. or skin temperature), but diffusion can be increased significantly when heated. For example, by incorporating excipients and/or adhesives with melting or glass transition temperatures above skin temperature (e.g., 32° C.) into the transdermal membrane 10, the membrane 10 matrix can allow diffusion of the drug from the transdermal membrane 10 to the skin upon heating. In one embodiment, small molecules or polymeric components with appropriate melting point or glass transition temperatures can be incorporated into the transdermal membrane 10. This is in contrast to common transdermal matrix systems, which typically deliver active agent as soon as applied to the subject according to their active agent concentration driven controlled-release kinetics.

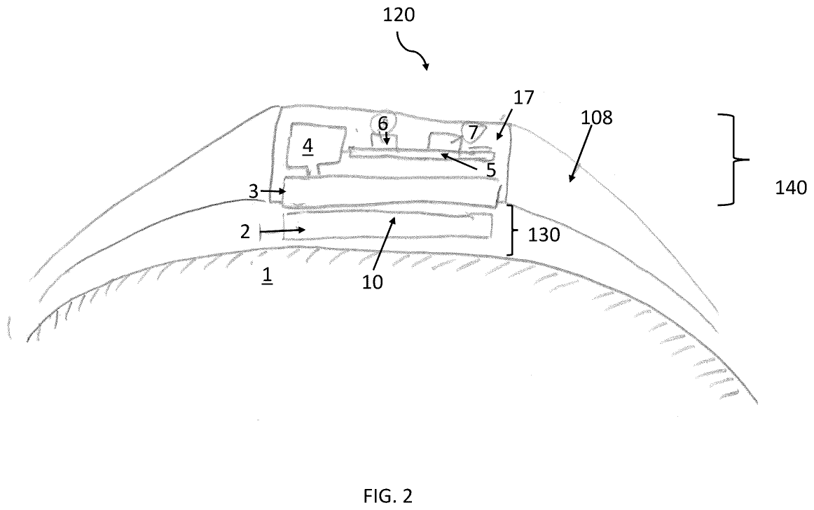

FIG. 2 shows another transdermal delivery system 120 with temperature control element 3 and transdermal membrane 10 containing active agent 2 for delivery to a subject. Transdermal delivery system 120 is similar to transdermal delivery system 20, but has two interconnectable parts 130, 140 and a band 108. The first part 130 includes transdermal membrane 10. The second part 140 includes the band 108 and housing 17 (which includes temperature control element 3, power source 4, flexible circuit board 5, microcontroller 6, and communication chip 7). The temperature control element 3 is configured to actively heat and/or actively cool a portion of the transdermal delivery device (e.g., transdermal membrane 10) as described herein. In some embodiments, the first part 130 can be disposable and the second part 140 can reusable.

As shown in FIG. 2, the band 108 can be connected to housing 17 and is configured to hold housing 17 close to a subject. Band 108 may be configured to wrap around a subject's body, such as an arm or leg to hold housing 17 close to the subject. In some embodiments, band 108 may hold both first part 130 and second part 140 against a subject's body. First part 130 may additionally include an adhesive (as described above) with respect to FIG. 1, which may hold transdermal membrane 10 against a subject's body. In some embodiments, first part 130 is separable from second part 140. For example, first part 130 and second part 140 may connect, such as through mating magnets on each part or mating snap-fit parts. In some other embodiments, first part 130 and second part 140 may be integral, e.g., not separable. In some embodiments, housing 17 may not contain all of the elements of the temperature control element 3, power source 4, flexible circuit board 5, microcontroller 6, and communication chip 7. For example, housing 17 may contain temperature control element 3 while communication chip 7 may be absent or may be contained in first part 130.

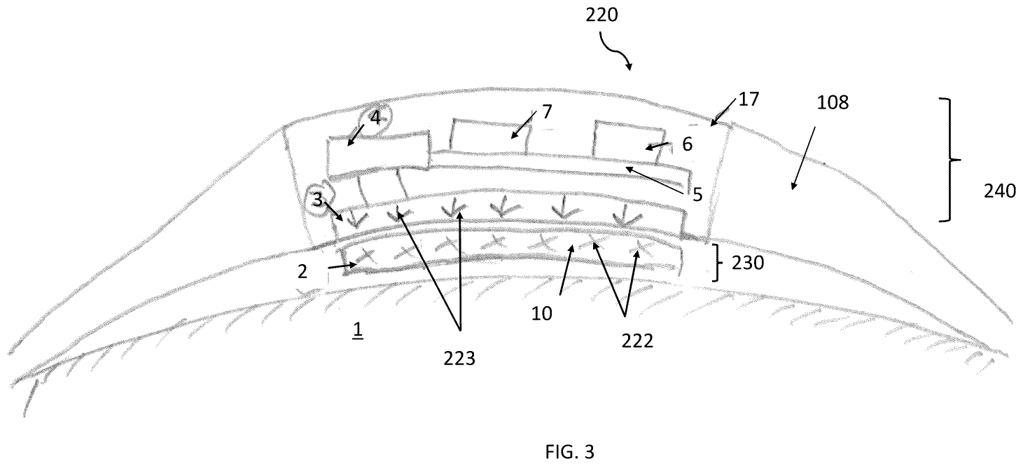

FIG. 3 shows another transdermal delivery system 220 useful for controlling active agent delivery to a subject. Delivery system 220 is similar to system 120 (and includes two parts 230, 240) except that the first part 230, which can be disposable, contains reactive material 222 to hold or otherwise prevent the active agent 2 from flowing across the transdermal membrane, and the second part 240, which can be reusable, is configured to produce stimulus 223 to act on the reactive material 222 and control the active agent 2 flow. The delivery system 220 thus includes the transdermal membrane 10 with reactive material 222 and active agent 2 and temperature control element 3 configured to provide a stimulus 223 to reactive material 222 to control active agent 2 flow. The reactive material 222 can be a plurality of small molecules, a solvent, or a polymeric material that is responsive to the stimulus 223 from the temperature control element 3. The reactive material 222 may include, for example, a polymer with a desired glass transition profile, a solvent, gold covered nanoparticles responsive to heat, and/or magnetic (e.g., iron) nanoparticles responsive to electromagnetic radiation. For example, reactive material 222 may be a polymer having a glass transition temperature (T_{g}) configured to transition from a first form to a second form at a desired temperature. In the first form, the polymer may have a hard, solid, and/or glassy state that prevents or minimizes active agent 2 flow and in the second form, the polymer may have a softer, more rubbery, and/or more viscous state that allows active agent flow. The stimulus 223 may be heat or other energy from the temperature control element 3 configured to transition the polymer from the first form to the second form. The reactive material 222 may be in a relatively hard, solid and/or glassy state at lower temperatures, such as at room temperature and/or skin temperature (e.g., at less than 32° C., less than 33° C., less than 34° C., or less than 35° C.). The reactive material 222 may be in a softer, more rubbery, and/or more viscous state at higher temperatures, such as above room temperature and skin temperature, (e.g., at least 34° C., at least 35° C., at least 36° C., at least 37° C., at least 38° C., at least 39° C., or at least 40° C.). The reactive material 222 may prevent substantial diffusion of the active agent 2 when the reactive material 222 is in the relatively hard, solid and/or glassy state at a lower temperature. The reactive material 222 may release or otherwise allow the active agent 2 to flow to the skin 1 of a subject when the reactive material 222 is in the softer, more rubbery, and/or more viscous state at a higher temperature. For example, the reactive material 222 may include an epoxy, a polyethylene, a polymethacrylate, a polypropylene, a polypropylene glycol, a polyvinylacetate, a polystyrene, a polytetrafluoroethylene, a poly (bisphenol A carbonate), a poly (ethylene terephthalate), a polylactic acid (PLA), a polyglycolic acid (PGA), and/or a polyurethane. The reactive material 222 may be tuned to have a desired glass transition temperature useful for holding and releasing a particular active agent in the transdermal system such as by including or excluding bulky, inflexible side groups, having relatively shorter or longer side chains, having a greater or lesser degree of crosslinking, or including or excluding a plasticizer such as nitrobenzene, B-napthyl salicylate, carbon disulphide, glycerine, propylene glycol, triethyl citrate, triacetine, polyethylene glycol, having greater or less hydrophobicity. In some examples, the reactive material 222 may include a polymeric hydrogel.

As indicated above, the delivery system 220 can include temperature control element 3 configured to deliver the stimulus 223 to reactive material 222 to control flow of active agent 2. The stimulus 223 from temperature control element 3 may be, for example, kinetic energy, a magnetic force, an electrical pulse, infrared radiation, or electromagnetic radiation. Temperature control element 3 may include chemicals (e.g., calcium chloride, iron particles, supersaturated sodium acetate), an electromagnet, an inductive coil, an infrared light source, phase change materials, and/or a visible light source to provide the stimulus 223.

In some variations, the stimulus 223 may be configured to act on the reactive material 222 such that the reactive material 222 holds or otherwise prevents active agent 2 from flowing across the transdermal membrane 10 to a subject. Turning the stimulus 223 off can allow a pulse of active agent 2 to flow across the transdermal membrane 10 to a subject. The cycle may be repeated multiple times to provide pulsatile active agent delivery, as described elsewhere herein.

In some embodiments, the transdermal system 220 may include two or more different types of reactive materials, and the transdermal system 220 may be configured to control pulsatile delivery of the active agent 2 or to control delivery of two different kinds of active agent 2. For example, a first type of reactive material 222 may have a first glass transition temperature (Tg) and may release its payload of active agent 2 at a first (lower) temperature (e.g., 36° C.) while a second type of reactive material 222 may have a second glass transition temperature (Tg) component and may release its payload of active agent 2 at a second (higher) temperature (e.g., 39° C.). Active agent delivery may be controlled by first delivering a first stimulus 223 to a portion of the transdermal system so the first type of reactive material 222 releases its payload of active agent 2, optionally turning the first stimulus 223 off, and delivering a second stimulus 223 to a portion of the transdermal system so the second type of reactive material 222 releases its payload of active agent 2. Such first and second payloads may release the same kind of active agent 2 (e.g., in a pulsatile manner) or may release different types of active agents.

Although the reactive material 222 may be in the transdermal membrane 10, it can additionally or alternatively be in another part of the transdermal delivery system 220 and may be configured to control pulsatile delivery. For example, the reactive material 222 can be in an active agent reservoir. The active agent reservoir can be separate from the transdermal membrane 10 and may be fluidically connected to it. The active agent reservoir may be configured to hold active agent 2. The stimulus 223 can act on the reactive material 222 in the active agent reservoir to release the active agent 2 from the active agent reservoir and into the transdermal patch. The stimulus 223 can allow release of active agent 2 from the active agent reservoir all at one time. Alternatively, the stimulus 223 can allow pulsatile release of active agent 2 from the active agent reservoir. An active agent reservoir may be in any of the delivery systems described herein, and may be in a first part or a second part of the delivery system.

In some variations, the active agent 2 can be the reactive material 222 responsive to stimulus 223, and there is can be no separate reactive material 222 that controls active agent flow through the transdermal membrane 10. In some embodiments, stopping stimulus 223 stops or significantly slows releasing, heating or another mechanism otherwise allowing active agent to flow across transdermal membrane 10. Significantly slowing the releasing, heating or other mechanism allowing active agent flow reduces the level of active agent flow relative to the “on” or stimulated level, such that even if some active agent is flowed across the transdermal membrane 10, it does not have the “on” effect and may be sufficiently low to have no therapeutic or detectable effect on the subject. In some embodiments, a stimulus 223 from temperature control element 3 may act on reactive material 222 to actively stop reactive material 222 from releasing or flowing active agent across transdermal membrane 10. For example, temperature control element 3 may include a heat sink, an endothermic ventilation fan, an evaporative based cooling mechanism, a sublimation based cooling mechanism, cyclic refrigeration, thermoelectric refrigeration, or magnetic refrigeration and may be configured to cool reactive material 222 and prevent active agent flow to a subject. In some embodiments, the temperature control element 3 may be configured to deliver both an “on” stimulus such as heat to turn on active agent flow across the transdermal membrane 10 and an “off” stimulus such as refrigeration to prevent or decrease active agent flow across the transdermal membrane 10.

Any of the transdermal systems described herein may have a discreet or low profile. Such a system may be worn with minimal notice by the user (or others) or with no or minimal discomfort while sleeping. In some embodiments, the transdermal system may be less than 50 cm³ in volume, less than 25 cm³ in volume, (e.g., 5 cm×10 cm×0.5 cm), less than 20 cm³ in volume, less than 15 cm³ in volume or less than 10 cm³ in volume. In some embodiments, the transdermal system may have dimensions that are less than 5 cm on a first side, less than 10 cm on a second side, and less than 0.5 cm on a third side. In some embodiments, the transdermal system may weigh less than 60 grams, less than 30 grams, less than 20 grams, less than 10 grams or less than 5 grams.

Any of the transdermal systems described herein may be very quiet (or silent) during use such as producing less than 20 dB, less than 10 dB, or less than 5 dB of sound during use.

In some embodiments, the transdermal systems may be held to a subject, such as by using the band 108 or strap that wraps around part of a user's body. The adhesive may be configured to adhere the transdermal system against the user's skin only using the adhesive. In some embodiments, the system may be sufficiently small, lightweight or flat such that the adhesive can readily hold the system in place on a user without the use of the band 108 or strap. The transdermal systems may be configured to be placed in any desired location on a user's body (e.g., abdomen, arm, back, leg, scalp, upper arm) using adhesive 14 and/or band 108.

Also described herein are methods for transdermally delivering an active agent. Some methods include actively heating or cooling a portion of a transdermal active agent delivery device for a first amount of time. Some methods include the step of after the first amount of time, actively heating or cooling the portion of the transdermal active agent delivery device for a second amount of time. In some such methods, the actively heating or cooling steps provide pulsatile delivery of active agent from a transdermal membrane of the transdermal active agent delivery device to skin of a patient. In some methods, the actively heating or cooling steps comprises delivering pulsatile heat to the portion of the drug delivery device. In some methods, the actively heating or cooling steps comprises removing heat from the portion of the transdermal active agent delivery device. In some methods, at least one of the actively heating or cooling steps comprises delivering electromagnetic radiation to the portion of the drug delivery device. Some methods include repeating the actively heating or cooling steps at least once. Some methods include repeating the actively heating or cooling steps at least two times. Some methods include repeating the actively heating or cooling steps at least three times. In some methods, the portion of the transdermal delivery system is heated by at least 4° C. Some methods include actively heating skin of the subject adjacent the transdermal membrane by at least 3° C. In some methods, at least one of the actively heating or cooling steps is performed while the subject is sleeping.

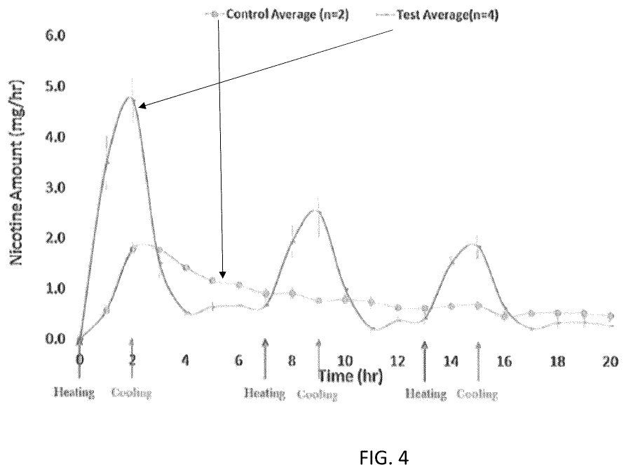

FIG. 4 shows results from using a transdermal delivery system as described herein with periodic heating and cooling. Experiments were carried out by applying passive matrix-type nicotine patches containing 21 mg of nicotine to human cadaver skin and then periodically heating and cooling the patches over a period of 20 hours. Controls with no heating and cooling were tested concurrently. The test patches were subject to 3 cycles of heating and cooling while the control patches were not. Patches were heated for 2 hours, cooled for 5 hours, heated for 2 hours, cooled for 4 hours, heated for 2 hours, and cooled. Nicotine delivery across the skin was measured and averaged for control patches (n=2) and test patches (n=4). Nicotine flow across the skin increased when heat was applied, and reached levels around 4.9 mg/hr, around 2.5 mg/hr, and around 1.8 mg/hr, respectively, during each of the three time periods when heat was applied. The level of nicotine delivery across the skin rapidly increased with heating and rapidly decreased with cooling as noted by the steep slope of increased nicotine flow after each heat application and steep decline of nicotine delivery after each cooling application. FIG. 4 shows that pulsatile active agent (nicotine) delivery was achieved by alternate cycles of heating and cooling.

Any of the systems or methods described herein, and in particular in a method for delivering an active agent to a subject, may include or be configured to include at least one of following or acceptable salts thereof, Acamprosate, Acetaminophen, Acetaminophen+Oxycodone, Alevicyn SG, Alfentanil, Allopurinol, Almotriptan, Alprazolam, Amitriptylinem, Amoxapine, Apomorphine, Aripiprazole, Armodafinil, Asenapinemaleate, Atomoxetine, Azelastine HCl, Baclofen, Benzbromarone, Benzydamine, Brexpiprazole, Budesonide, Bupivacaine, Buprenorphine, Buprenorphine+Nalaxone, Bupropion, Bupropion Hydro bromide, Bupropion Hydrochloride, Buspirone, Cabergoline, Capsaicin, Carbamazepine, Carbidopa+Levodopa, Carisprodol, Celecoxib, Citalopram, Clobazam, Clonazepam, Clonidine, Clopidogrel, Colchicine, Cyclobenzaprine, Dalteparin sodium, Desvenlafaxine, Dexamfetamine, Dexmethylphenidate HCl, Diazepam, Diclofenac, Diclofenac Potassium, Disulfiram,Divalproex Sodium, Dolasetron Mesilate, Doxepin, Dronabinol, Droxidopa, Duloxetine, Eletriptan, Entacapone, Escitalopram oxalate, Eslicarbazepine Acetate, Esomeprazole/naproxen, Estradiol, Estrogen, Eszopiclone, Ethosuximide, Etodolac, Ezogabine, Febuxostat, Felbamate, Fenbufen, Fentanyl, Fentanyl Citrate, Fentanyl HCl Flunisolide, Fluorouracil, Fluoxetine, Fluticasone propionate, Fluvoxamine, Formoterol, Fosphenytoin, Frovatriptan, Gabapentin, Granisetron, Guanfacine, Hydrocodone Bitartrate, Hydrocodone+Acetaminophen, hydrocortisone, Hydromorphone Hcl, Hydroxyzine, Hypericum Extract, Ibuprofen, Indometacin, Ketorolac, Lacosamide, Lamotrigine, Levetiracetam, Levomilnacipran, Levosalbutamol, Lidocaine, Lidocaine/Tetracaine, Lisdexamfetamine, Lithium Carbonate, Lorazepam, Lorcaserin Hydrochloride, Losartan, Loxapine, Meclizine, Meloxicam, Metaxalone, Methylphenidate, Methylphenidate Hydrochloride, Milnacipran, Mirtazapine, Modafinil, Morphine, Nabilone, Nadolol, Naltrexone, Naproxen, Naratriptan, Nedocromil, Nefazodone, Nicotine, Nicotine Sulfate, Nicotine salts, Nitroglycerin, Olanzapine, Ondansetron, Orlistat, Oxaprozin, Oxcarbazepine, Oxybutynin, Oxycodone, Oxycodone+Acetaminophen, Oxycodone Hydrochloride, Oxymorphone, Palonosetro, Pamidronate, Paroxetine, Paroxetine Mesylate, Perampanel, Phentermine+Topiramate, Phentermine Hydrochloride, Phentolamine Mesylate, Pramipexole, Prasugrel, Prazepam, Prednisone, Pregabalin, Promethazine, Propofol, Quetiapine, Quetiapine Fumarate, Ramelteon, Rasagiline, Rasagiline Mesylate, Remifentanil, Risperidone, Rivastigmine, Rivastigmine Tartrate, Rizatriptan, Ropinirole, Ropivacaine, Rotigotine, Rufinamide, Salbutamol, Scopolamine, Selegiline, Sertraline, Sodium Oxybate, Strontium, Sufentanil, Sumatriptan, Sumatriptan Succinate, Suvorexant, Tapentadol, Tasimelteon, Temazepam, Testosterone, Tetracaine+Lidocaine, Theophylline, Tiagabine, Tiotropium, TirofibanHCI, Tolcapone, Topiramate, Tramadol, Tramadol+Acetaminophen, Trazodone, Triazolam, Trimipramine Maleate, Valproate Semi sodium, Valproate Sodium, Venlafaxine, Vigabatrin, Vilazodone, Vortioxetine, Zaleplon, Zileuton, Ziprasidone, Zolmitriptan, Zolpidem, Zolpidem Tartrate, Norethisterone Acetate (NETA), Enapril, Ethinyl Estradiol, Insulin, Memantine, Methamphetamine, Norelgestromine, Pergolide, Ramipril, Tecrine, Timolol, Tolterodine and Zonisamide.

In some particular examples, the systems or methods described herein may be configured to aid in smoking cessation or to treat aspects of Parkinson's Disease. For example, in some particular examples, the systems or methods described herein may include nicotine, a nicotine analog, a nicotine antagonist, a nicotine agonist, benztropine (Cogentin), carbidopa, dopamine, a dopamine analog, a dopamine antagonist, a dopamine agonist, entacapone, levodopa (L-dopa), pramipexole (Mirapex), rasagiline (Azilect®), ropinirole (Requip), rotigotine (Neupro®), safinamide (Xadago), selegiline (Eldepryl®, Zelapar™), sinemet (both carbidopa and levodopa), and trihexyphenidyl (Artane®), and tolcapone (Tasmar).

The active agent 2 may be in the form of a solution, a suspension, a gel, or a dispersion. In some embodiments, the transdermal delivery system contains the active agent 2. In some embodiments, a transdermal delivery system as described herein may be replenished with active agent from an outside source and in some embodiments, a transdermal delivery system as described herein may not contain an active agent and may be configured to receive added active agent. For example, an active agent may be added to the transdermal delivery system by injection of active agent into the transdermal delivery system (e.g., into the transdermal membrane) or by adding an active agent reservoir configured to flow active agent to the delivery system.

It should be understood that features described with respect to one embodiment may be substituted for or used in addition to features described with respect to another embodiment.

The systems and methods described herein can further include any of the elements or steps described in U.S. Pat. Nos. 8,673,346, 9,555,277, 8,372,040, 10,105,487, 10,213,586, U.S. Publication No. 2018/0374381, PCT Publication No. WO2018/106723, PCT Publication No. WO2018/129304, PCT Publication No. WO2018/148746, PCT Publication No. WO2018/129363, U.S. Publication No. US2018/0110768, or U.S. Publication No. 2019/0054078, the entireties of which are incorporated by reference herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A transdermal delivery system comprising:
a transdermal membrane configured to contact a skin of a subject;
a first active agent fluidically connected to the transdermal membrane, the transdermal membrane configured to control, in response to a first temperature, a flow of the first active agent to the skin of the subject; and
a second active agent fluidically connected to the transdermal membrane, wherein the transdermal membrane is further configured to control, in response to a second temperature, a flow of the second active agent to the skin of the subject; and
a temperature control element configured to cyclically heat to a first temperature and/or cool a portion of the transdermal delivery system so as to provide pulsatile delivery of the first active agent through the transdermal membrane, and configured to cyclically heat to a second temperature, different from the first temperature, the portion of the transdermal delivery system so as to provide pulsatile delivery of the second active agent through the transdermal membrane.

2. The transdermal delivery system of claim 1, wherein the first active agent is contained in a reservoir separate from the transdermal membrane.

3. The transdermal delivery system of claim 1, wherein the temperature control element comprises an electromagnetic energy source.

4. The transdermal delivery system of claim 1, wherein the temperature control element comprises a resistive element.

5. The transdermal delivery system of claim 1, wherein the temperature control element comprises an inductive coil.

6. The transdermal delivery system of claim 1, wherein the temperature control element comprises a coolant or heat sink.

7. The transdermal delivery system of claim 1, wherein the portion of the transdermal membrane comprises a polymer configured to be heated or cooled to thereby change the first or the second active agent flow.

8. The transdermal delivery system of claim 1, wherein the portion of the transdermal membrane comprises a glass transition polymer configured to be heated or cooled to thereby change the first or the second active agent flow.

9. The transdermal delivery system of claim 1, wherein the portion of the transdermal membrane comprises a magnetic nanoparticle configured to be heated or cooled to thereby change the first or the second active agent flow.

10. The transdermal delivery system of claim 1, wherein the first active agent comprises nicotine or a nicotine agonist.

11. The transdermal delivery system of claim 1, wherein the active agent comprises a Parkinson's disease treatment.

12. The transdermal delivery system of claim 1, further comprising an adhesive in the transdermal membrane configured to adhere the transdermal membrane to the subject.

13. A method for transdermally delivering active agents to a subject, comprising:
fluidically coupling a first active agent to a transdermal membrane;
fluidically coupling a second active agent to the transdermal membrane;
cyclically heating to a first temperature and cooling a portion of a transdermal delivery system for a first amount of time to provide pulsatile delivery of the first active agent through the transdermal membrane; and
after the first amount of time, cyclically heating to a second temperature and cooling the portion of the transdermal delivery system for a second amount of time to provide pulsatile delivery of the second active agent through the transdermal membrane.

14. The method of claim 13, wherein the first active agent is included in a reservoir fluidically connected to the transdermal membrane.

15. The method of claim 13, wherein the cyclic heating and cooling is provided by a temperature control element.

16. The method of claim 13, wherein the cyclic heating and cooling comprise delivering heat to the portion of the transdermal membrane.

17. The method of claim 13, wherein the cyclic heating and cooling comprises removing heat from the portion of the transdermal membrane.

18. The method of claim 13, wherein the cyclic heating and cooling is performed while the subject is sleeping.

19. The method of claim 13, further comprising repeating the cyclic heating or cooling steps at least once.
